(19) United States
(12) Patent Application Publication
(10) Pub. No.: US 2018/0035932 A1
(43) Pub. Date: Feb. 8, 2018

(54) METHOD AND APPARATUS FOR REAL-TIME NON-INVASIVE OPTICAL MONITORING OF DECOMPRESSION SICKNESS STATE

(71) Applicant: Irina Massova, Belmont, CA (US)

(72) Inventor: Irina Massova, Belmont, CA (US)

(21) Appl. No.: 15/230,450

(22) Filed: Aug. 7, 2016

(51) Int. Cl. A61B 5/1455; A61B 5/08; B63C 11/12; A61B 5/024; A61B 5/00; A62B 9/00

(52) U.S. Cl. CPC A61B 5/14551; A61B 5/6803; A62B 9/00; B63C 11/12; A61B 5/02416; A61B 5/0816

(57) ABSTRACT

Described herein is a device and system and method for the detection of naturally occurring gas bubbles in the bloodstream; in one embodiment, a wireless optical device may be placed on various parts of the body including a finger or earlobe or is coupled with the SCUBA mouthpiece and detects certain vital signs such as heart rate, blood oxygen saturation (SpO2) and respiration rate by analyzing the photoplethysmogram (PPG) from a miniature sensor. There is also a method and apparatus for real-time non-invasive optical monitoring of actual state of decompression sickness are presented. The method is based on simultaneous measurements of PPG signals in different locations of human body and analysis of such a factor as phase shifts between various locations, change the shape of PPG signal between different locations, existence and modification of slow trends and low frequency modulations and angular distributions of the out-coming light.

### CROSS REFERENCE TO RELATED APPLICATIONS

This application claims the benefit of U.S. Provisional Patent Application No. 61/940,482, filed Feb. 16, 2014, titled “A DEVICE FOR OPTICAL MEASURING OF DECOMPRESSION SICKNESS OF A DIVER,” U.S. Provisional Patent Application No. 61/976,321, filed Apr. 7, 2014, titled “METHOD AND APPARATUS FOR REAL-TIME NON-INVASIVE OPTICAL MONITORING OF DECOMPRESSION SICKNESS STATE,” and International Application PCT PCT/US2015/015964 “METHOD AND APPARATUS FOR REAL-TIME NON-INVASIVE OPTICAL MONITORING OF DECOMPRESSION SICKNESS STATE,” each of which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Embodiments of the invention relate to the detection of naturally occurring gas bubbles in the bloodstream. More particularly, the present invention relates to an optical device to detect Caisson's disease (decompression sickness) in situations where gas bubbles are released in body tissues and fluids upon a too rapid decrease in surrounding pressure after the body's stay in a compressed atmosphere.

Additional aspects relate generally to the field of non-invasive monitoring of decompression sickness (DCS) and relates to a method and apparatus for determining the actual fraction of gas phase in a diver's blood. In still other aspects, there are systems and methods for the monitoring of DCS related symptoms like cognitive distortion.

### BACKGROUND

Decompression sickness (DCS) is an umbrella term covering pathophysiological changes and problems resulting from drastic reduction in external pressure. Decompression associated risks are definitely among the dominant ones in diving, aeronautics, mountain climbing etc. The basic widely accepted mechanism of the DCS is reduced to the following logical chain of processes: fast reduction of ambient pressure leads to decrease of inertial gas solubility that favors formation and growth of gas bubbles and multiple cases of vessel embolism.

The actual challenge is to measure non-invasively the real-time fraction of the gas phase in blood both in the process of diving and in the post-diving period.

Existing techniques utilize a handheld or portable ultrasound device comprising a transducer propagating and receiving sound signals to/from a blood vessel and a controller receiving the sound signals from the transducer to determine/monitor presence of naturally occurring bubbles in the blood vessel because of decompression sickness. Many existing devices require a significant power source. As a result, there is a need for a smaller, energy efficient device that would that provide the above features and offer additional vital measurements.

### SUMMARY OF THE DISCLOSURE

In general, in one embodiment, a method of determining an initiation or progression of decompression sickness in an individual using an in-vivo measurement of time-dependence of optical density of at least one irradiated body part, the method including irradiating a portion of at least one body part with a photoplethysmography (PPG) sensor; collecting a time dependent optical signal from the sensor; and analyzing the collected system to determine the initiation or progression of decompression sickness in the individual.

This and other embodiments can include one or more of the following features. In one aspect, the irradiating step can be performed using a light in the yellow-green region. In another aspect, the irradiating step can be performed using a light in the near infrared region. In a further aspect, the analyzing step can be performed to determine an indicia of an optical scattering characteristic indicating the presence of a gas. In an alternative aspect, the method can further include irradiating another portion of a body part spaced apart from the portion of at least one body part and the analyzing step can be performed by comparing a blood velocity measurement at the portion and the another portion to determine an indicia of the presence of a gas influencing blood velocity. In yet another aspect, the irradiated portions can be spaced apart by a distance of 6 inches to 12 inches. In still another aspect, the portion or the another portion of at least one body part can be within the mouth, on a lip, on a cheek, on a forehead, on a face, on an ear, on a wrist, on an elbow, on an arm, on a leg, on a finger or on a hand. In one aspect, the irradiating step or the collecting step can be performed using a PPG sensor wherein at least one element of an emitter or a detector can be mounted on, in or within a respirator or SCUBA mouth piece or a diving mask. In another aspect, the irradiating step or the collecting step can be performed using a PPG sensor configured as one emitter and multiple detectors. In a further aspect, the irradiating step or the collecting step can be performed using a PPG sensor configured as one detector and multiple emitters. In an alternative aspect, the method performed using at least one PPG sensor can be configured for transmission mode operation. In yet another aspect, the method performed using at least one PPG sensor can be configured for reflectance mode operation.

In general, in one embodiment, a PPG sensor enabled diving mask, includes a face piece having a lens and a seal or skirt; and at least one PPG sensor on, in or within a portion of the seal or the skirt such that in use on a diver the at least on PPG sensor is positioned to irradiate a desired portion of a region of blood perfusion on the diver.

This and other embodiments can include one or more of the following features. In one aspect, the at least one PPG sensor can be adapted and configured to irradiate a desired portion using a light in the yellow-green region. In another aspect, the at least one PPG sensor can be adapted and configured to irradiate a desired portion using a light in the near infrared region. In a further aspect, the at least one PPG sensor can be configured as one emitter and multiple detectors. In an alternative aspect, the at least one PPG sensor can be configured as one detector and multiple emitters. In yet another aspect, the at least one PPG sensor can be configured for transmission mode operation. In still another aspect, the at least one PPG sensor can be configured for reflectance mode operation.

In general, in one embodiment, a PPG sensor enabled respirator mouthpiece, includes a respirator mouthpiece having a connector to attachment to a respirator supply and a portion for placement within the mouth of a user having an inner surface and an outer surface; and at least one PPG sensor on, in or within a portion of the respirator mouthpiece positioned to irradiate a desired portion of a region of blood perfusion on the user having the mouthpiece in his mouth.

This and other embodiments can include one or more of the following features. In one aspect, the at least one PPG sensor can be adapted and configured to irradiate a desired portion using a light in the yellow-green region. In another aspect, the at least one PPG sensor can be adapted and configured to irradiate a desired portion using a light in the near infrared region. In a further aspect, the at least one PPG sensor can be configured as one emitter and multiple detectors. In an alternative aspect, the at least one PPG sensor can be configured as one detector and multiple emitters. In yet another aspect, the at least one PPG sensor can be configured for transmission mode operation. In still another aspect, the at least one PPG sensor can be configured for reflectance mode operation.

In general, in one embodiment, a method of real-time non-invasive monitoring of the actual state of decompression sickness, the method including the steps of: obtaining reference data indicative of the spectral behavior of time-variant optical response modulated by heartbeats of diver; irradiating any part of diver's body with at least one light source with the wavelength belonging to the red-near infrared (RNIR) spectral range or the yellow green spectral range; measuring the optical response by at least one optical sensor of the same wavelength to obtain a first set of photoplethysmographic (PPG) data; performing the irradiating and the measuring steps with at least one additional light source and optical sensor to obtain a second set of photoplethysmographic (PPG) data; and combining said first set and said second set of photoplethysmographic (PPG) data from at least two different PPG units obtaining PPG information from different locations on the body.

This and other embodiments can include one or more of the following features. In one aspect, the said at least two PPG units or the first and second set of data can be synchronized with the common clock. In another aspect, the method can further include comparing the first and the second data from the at least two said different PPG units to measure a phase shift in the PPG signal between two different locations on the body. In a further aspect, the method can further include analyzing the PPG data to determine changes in the relative amplitudes of the main peak “1” and secondary peak “3” of said PPG signals between said different locations on the body. In an alternative aspect, at least one PPG data collection unit can operate with reflected or/and back-scattered light. In yet another aspect, at least one PPG data collection unit can operate in a transmission mode. In still another aspect, at least one PPG data collection unit can operate in a reflectance mode. In one aspect, the measurements and analysis can be performed by said modified PPG unit having more than one detector optically coupled to the same light source. In another aspect, the analyzing and measuring the data where at least two of said optical detectors belonging to the same PPG unit can have different distance from the light source of the same modified PPG unit. In a further aspect, said measurements can be performed during a time interval exceeding the period of one heartbeat of a diver. In an alternative aspect, said measurements can be performed for multi-period PPG or providing for measuring to the slow trend of PPG. In yet another aspect, said measurements and further analysis can define one or more slow frequency components when their frequencies are lower than the frequency of heartbeats. In still another aspect, when the existence of a measured slow frequency can be a basis or indication of a cognitive distortion caused by decompression sickness. In one aspect, a measured slow trend cannot be periodic or can be non-repeating or can have an irregular occurrence. In another aspect, the measured slow trends can relate to PPG or oximetry data taken at the same depth of a diving event or can be related to collection during the process of descent or can be related to collection during the process of ascent and thereafter one or more of the measured slow trends can be compared. In a further aspect, the measured slow trends can deal with any PPG data taken at the same depths but in the processes of descent and ascent and then compared. In an alternative aspect, the method can further include determining an actual state of decompression sickness based on the comparison steps. In yet another aspect, the method can further include comparing between the optical signals of two sets of light detectors coupled to the same light source and tuned to the same wavelength but can have different distances from the said light source for obtaining an angular distribution of the out-coming light thus providing information about the existence of additional centers of light scattering in the diver's blood. In still another aspect, the method can further include synchronizing to a common clock the one or the more than one PPG units can be used for performing any of the steps located at different points of diver's body. In one aspect, there can be at least one modified PPG unit having several optical sensors coupled optically to the same light source and the sensors can be located at different distances from the same light source and the method steps can be performed using the PPG data collected from this PPG unit configuration. In another aspect, the wavelength of light used to perform a step or provided in a PPG sensor can be within the range of 300 nm to 1,300 nm or is 660 nm or is 940 nm in any combination.

In general, in one embodiment, a method of real-time non-invasive monitoring of the actual state of decompression sickness including the steps of: providing reference data indicative of the spectral behavior of time-variant optical response modulated by heartbeats of diver; irradiating any part of diver's body with at least one light source with the wavelength belonging to the red-near infrared (RNIR) spectral range and measuring the optical response by at least one optical sensor of the same wavelength to get a photoplethysmographic (PPG) signal, and creating a basic configuration “light source-light detector” of the same wavelength that is further called the “PPG unit” or “light source-several light detectors” of the same wavelength that is further called the “modified PPG unit”; combining said photoplethysmographic (PPG) data from at least two different PPG units located in different points of the body; keeping said at least two PPG units synchronized with the common clock; comparing the data from at least two said different PPG units to measure the phase shift between two different points of the human body; and analyzing the data for changes in the relative amplitudes of the main peak “1” and secondary peak “3” of said PPG signals between said different locations on the body.

This and other embodiments can include one or more of the following features. In one aspect, at least one of said PPG units or modified PPG units can work with reflected or/and back-scattered light. In another aspect, at least one of said modified PPG units can have more than one detector optically coupled to the same light source. In a further aspect, at least two of said optical detectors belonging to the same PPG unit can have different distance from the light source of the same modified PPG unit. In an alternative aspect, a time interval can exceed the period of one heartbeat of a diver. In yet another aspect, a multi-period PPG can measure the slow trend of PPG. In still another aspect, said measurement and further analysis can define one or more slow frequency components when their frequencies are lower than the frequency of heartbeats. In one aspect, the existence of slow frequency can be the basis to suspect the cognitive distortion caused by DCS. In another aspect, the measured slow trend cannot be periodic. In a further aspect, the measured slow trends can deal with oximetry data taken at the same depths but in the processes of descent and ascent and then compared. In an alternative aspect, the measured slow trends can deal with any PPG data taken at the same depths but in the processes of descent and ascent and then compared. In yet another aspect, the basis of said comparison the conclusions can be drawn on the actual state of DCS. In yet another aspect, the comparison between the optical signals of two sets of light detectors can be coupled to the same light source and tuned to the same wavelength but can have different distances from the said light source which can reveal the angular distribution of the out-coming light an can inform about the existence of additional centers of light scattering in the diver's blood. In still another aspect, the apparatus can include

several “PPG units” located at different points of diver's body and synchronized with the common clock. In a further aspect, the apparatus can include at least one modified PPG unit of the sort “several optical sensors coupled optically to the same light source” located at different distances from the same light source.

There is provided herein methods of and photoplethysmographic apparatus for monitoring or detecting the onset or degree of decompression sickness collecting and analyzing PPG data for gas fraction induced variations to blood scattering, absorption and/or velocity.

In one aspect, there is a comparison of PPG signal from different locations on a diver's body. Such an approach permits a system to simultaneously to analyze all kinds of phase shifts between typical single-period PPG at different locations. This phase shift reflects the changes in the arterial pulse wave velocity when the blood gas phase becomes significant. in some apparatus configurations there is included PPG sensors operating in reflection geometry. In some aspects, all PPG sensors are synchronized with the common clock.

Alone or in combination with these phase shifts, comparisons of the actual manifestations of basic and secondary peaks in different locations of diver's body that are indicators of some cases of small vessel embolism.

In still other aspects, there is provided a real-time picture of gas bubbles distribution monitored by the change in the angular distribution of the out-coming light. It also can be done in the reflective geometry of PPG sensors. In one aspect, the PPG sensors of an apparatus have several light detectors optically bound to the same light source but placed in different distances from this light source. The comparison of PPG measured by these sensors is translated into the angular distribution of light, and thus contains information about the existence of the additional scatterers in blood like gas bubbles.

In still another aspect of a method and apparatus is adapted and configured for multi-period PPG signal processing. In this aspect, there are steps for analyzing the slow trend phenomena of measured PPG. In one aspect the steps may be subdivided into two groups and each may be monitored for non-periodic slow trends in PPG signals in alike though non-identical conditions. For instance, the conditions may be those of a diver during one or several different phases of a diving event (i.e., surfaced, descent, at depth, at different depth, and or ascent). In one aspect, blood oximetry data are compared for the same depths but the one in the processes of descent with the oximetry data in the process of ascent. As the time-derivative of ambient pressure has the opposite signs, the amount of gas phase differs in these cases, so will the measured oximetry data. The second group of measurements deals with low frequency slow modulation of PPG signals. The existence of clear low frequency component is indicative of certain sleep phases like REM-sleep phase. As the electroencephalography (EEG) research show resemblance between EEG of post-diving period and the EEG of REM-sleep phase, in some aspects the method or system is configured to perform the same procedure with the PPG signals obtained as described herein.

### BRIEF DESCRIPTION OF THE DRAWINGS

The above and other objects, features, and advantages of the embodiments of the present invention are further described in the detailed description which follows, with reference to the drawings by way of non-limiting exemplary embodiments of the present invention, wherein like reference numerals represent similar parts of the present invention throughout the several views and wherein: with reference to the accompanying drawings, in which:

FIG. 1 shows the typical single period PPG signal of healthy person where one can easily identify both: the major peak 1, and the secondary peak 3, and the deep 2 between them. Timing of these features depends on the location of the PPG unit on the body, and the measured phase shift is the difference between corresponding features in different locations.

FIG. 2 demonstrates the typical multi-period PPG feature measured by our system. The presence of low frequency modulation and its expression is suggested in this invention as an indication of risk of cognitive distortions.

FIG. 3A demonstrates the layout of the one-wavelength part of our modified PPG unit. Such a layout provides in reflection the optical coupling between the light source and two sets of light detectors. All the detectors of each particular set, either the first set (detectors LD 1.1-LD1.4) or the second set (detectors LD1.5-LD1.8) have the same distance from the light source LS, while these distances for the first set and for the second set of detectors differ considerably.

FIG. 3B demonstrates the layout of the one-wavelength part of a modified PPG unit. Such a layout provides in reflection the optical coupling between a single optical detector and two or more sets of optical light sources. All the light sources of each particular set, either the first set (sources LS 1_1-LS 1_4) or the second set (sources LS 1_5-LS 1_8) have the same distance from the light detector, while these distances for the first set and for the second set of sources differ.

FIG. 4 is flow diagram showing the signal path from the optical transmitter to the CPU

FIG. 5 is a perspective view of a respirator or SCUBA mouthpiece showing seven different exemplary PPG sensor mounting or integration locations.

FIG. 6 is a perspective view of a respirator or SCUBA mouthpiece showing six different exemplary PPG sensor mounting or integration locations.

FIG. 7 is a perspective view of a respirator or SCUBA mouthpiece showing eight different exemplary PPG sensor mounting or integration locations.

FIG. 8 is a rear view of the sealing rim of a dive mask showing six different exemplary PPG sensor mounting or integration locations.

FIG. 9 is a view of an arm of a diver show two spaced apart PPG sensor locations on the arm between about 6 inches to 12 inches apart. In the illustrated embodiment, there is one sensor near the wrist and another sensor near the elbow.

### DETAILED DESCRIPTION

On one aspect, there is provided embodiments of devices, systems and methods for a detecting naturally occurring gas bubbles in the human bloodstream. In one embodiment, PPG signals are analyzed for determining or detecting the onset of or progression of or degree of decompression sickness (i.e., caisson's disease). In another aspect, PPG sensor data is analyzed for optical variations and mechanical variations as part of the method of determining the onset of or progression of or degree of decompression sickness (i.e., caisson's disease).

In one aspect, there is a method and a photoplethysmographic apparatus of a special design in order to perform such a monitoring. In one aspect, multiple PPG sensors are used in combination and may also be linked to a diver's watch or device for determining dive time overall, time at depth, descent rate, ascent rate and other parameters of a diving event in combination with one or more signals collected from one or more PPG sensors during the descent, dive duration or ascent phases of a scuba diving event. In various different configurations, there may be provided a variety of different means of communication between the PPG sensor(s) and a dive computer such as, for example, stand means used for communications in industrial diving operations, or radio system, wired configuration, via ultrasound transmission mode or other communication means suited to the underwater and diving environment. In still another aspect, PPG data collected during descent phase is compared to PPG data collected during ascent phase as part of a method of determining or detecting the onset of or progression of or degree of decompression sickness (i.e., caisson's disease). PPG sensors may be placed in any location suited to measuring blood perfusion based on the activity being undertaken. Exemplary areas for sensor placement would be on the head such as forehead or the cheeks, in the mouth, on a finger or on the chest, to name a few.

Photoplethysmography (PPG) is the in-vivo measurement of time-dependence of optical density of irradiated body part. This optical density quasi-periodically oscillates with the heartbeats following an oscillating behavior of arterial blood. The amplitudes of oscillations are wavelength dependent, and the resulting changes may be reliably observed in the red-near infrared (RNIR) spectral range. The actual magnitude of the PPG signal in the RNIR spectral range depends crucially both on absorption of blood key ingredients and on light scattering from blood. The time dynamics of the PPG signal depends also on the velocity of the pulse wave in large arteries. The existence of gas fraction in blood crucially changes both the scattering and the absorption of blood. Besides, it also changes the pulse wave velocity. Thus, the actual state of DCS can be monitored by PPG techniques if certain modifications of this technique are applied.

Generally, such a monitoring system (also known as a photoplethysmograph) includes a transmitter utilizing a probe attached to a part of the body (e.g., a finger, forehead, ear pinna or an earlobe) that includes an optical source, e.g., a light emitting diode (LED) or a laser, for irradiating the body part with light and a receiver utilizing an optical photodetector (e.g., a photo diode) positioned in an optical path so that it has a field of view which ensures the capture of a portion of the light which is transmitted, reflected or scattered from the body part. Resulting PPG signal depends on wavelength of the optical signal, refractive index and absorption coefficients of blood serum, red blood cells, water and hemoglobin. Additional details of PPG sensors and signal processing are provided in “Wearable Photoplethysmographic Sensors—Past and Present” by T. Tamura, Y. Meada, M. Sekine, and M. Yoshida in Electronics 2014, 3, 282-302; doi:10.3390/electronics3020282, published on 23 Apr. 2014, incorporated herein by reference for all purposes.

Given that both refractive index and absorption coefficient of blood serum will be modified by the presence or existence or increasing size or presence or existence of gas bubbles, as well as on the volume, shape and concentration of gas bubbles present. As a result, the processing methods and systems described herein are adapted and configured to advantageously process the resulting PPG sensor signals for the presence of or indicia of the presence of gas or bubbles in order to detect or determine the actual stage of decompression sickness. In one aspect, there is provided a method and a photoplethysmographic apparatus used in combination in order to perform such a monitoring.

Described herein is a device and system and method for the detection of naturally occurring gas bubbles in the bloodstream; in one embodiment, a wireless optical device may be placed on various parts of the body including a finger or earlobe or is coupled with the SCUBA mouthpiece and detects certain vital signs such as heart rate, blood oxygen saturation (SpO2) and respiration rate by analyzing the photoplethysmogram (PPG) from a miniature sensor.

Photoplethysmography (PPG) is the in-vivo measurement of time-dependence of optical density of irradiated body part. In some embodiments, PPG signals may be obtained using one or more or combinations of either reflection or transmission geometry configurations between the optical emitter and detector. Optical density quasi-periodically oscillates with the heartbeats following an oscillating behavior of arterial blood. The amplitudes of oscillations are wavelength dependent, and the resulting changes may be reliably observed in the red-near infrared (RNIR) spectral range. The actual magnitude of the PPG signal in the RNIR spectral range depends both on absorption of blood key ingredients and on light scattering from blood. The time dynamics of the PPG signal depends also on the velocity of the pulse wave in large arteries. The existence of gas fraction in blood crucially changes both the scattering and the absorption of blood. Besides, it also changes the pulse wave velocity. Thus, the actual state of DCS can be monitored utilizing embodiments of the modified PPG techniques described herein.

First, in one aspect there is a method for a comparison of PPG signal from different PPG sensors located on a diver's body. Such an approach enables simultaneous real time analysis of a variety of phase shifts between the single-period PPG signals collected from the different locations. This phase shift corresponds to the changes in the arterial pulse wave velocity when the blood gas phase becomes significant. In one embodiment, there is an apparatus that includes one or more PPG sensors operating in reflection geometry. In one configuration, the sensors are synchronized with a common clock of the signal processing system.

Alone or in combination with the phase shifts, aspects of the system processing PPM data also detects the actual manifestations of basic and secondary peaks in the different locations of diver's body. In one aspect, the basic and the secondary peaks and comparisons are indicative on the cases of small vessel embolism.

In another aspect, the real-time characterization of gas bubble distribution is also detected and/or monitored by the change in the angular distribution of the out-coming light. Additionally or alternatively, angular light distribution may also be performed in a PPG sensor reflective geometry configuration. In this aspect, the PPG sensors of our apparatus have several light detectors in varied spatial arrangement and distances but optically bound to the same light source. The comparison of PPG measured by these sensors is translated into the angular distribution of light. The angular distribution of light thus contains information about the existence of the additional optical scatterers in blood like gas bubbles.

In still another aspect of an embodiment of our method and apparatus deals not with single-period PPG signal but with a multi-period signal. This analysis is used for the slow trend phenomena of measured PPG. This analysis is subdivided into two groups with non-periodic slow trends in PPG signals in alike though non-identical conditions. In one exemplary embodiment, blood oximetry data are compared for the same dive depths but the one in the processes of descent with the oximetry data in the process of ascent. As the time-derivative of ambient pressure has the opposite signs, the amount of gas phase differs in these cases, so the measured oximetry data also does. The second group of measurements deals with low frequency slow modulation of PPG signals. The existence of clear low frequency component as our studies show is indicative of certain sleep phases like REM-sleep phase. As the electroencephalography (EEG) research show resemblance between EEG of post-diving period and the EEG of REM-sleep phase, in one embodiment the same procedure is performed with the PPG signals.

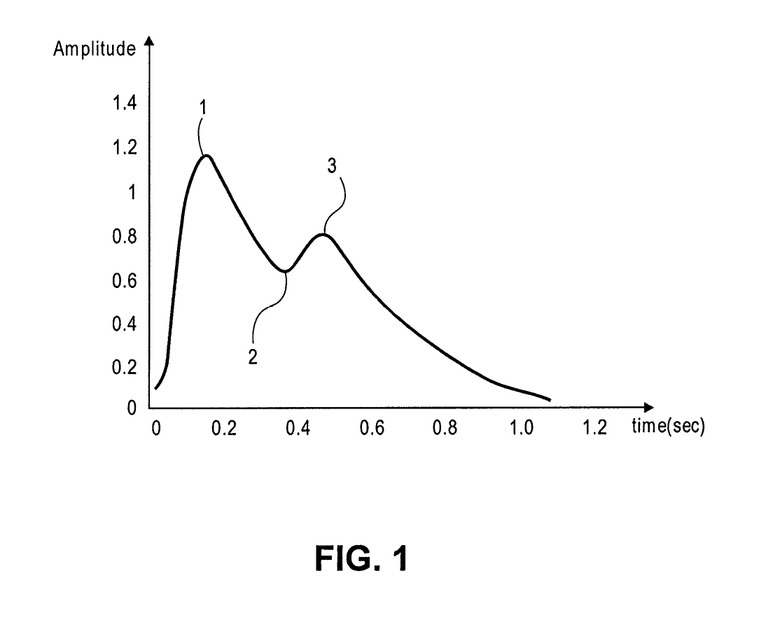

FIG. 1 shows the typical single period PPG signal of healthy person where one can easily identify both: the major peak 1, and the secondary peak 3, and the deep valley 2 between them. Timing of these features depends on the location of the PPG unit on the body, and the measured phase shift is the difference between corresponding features in different locations.

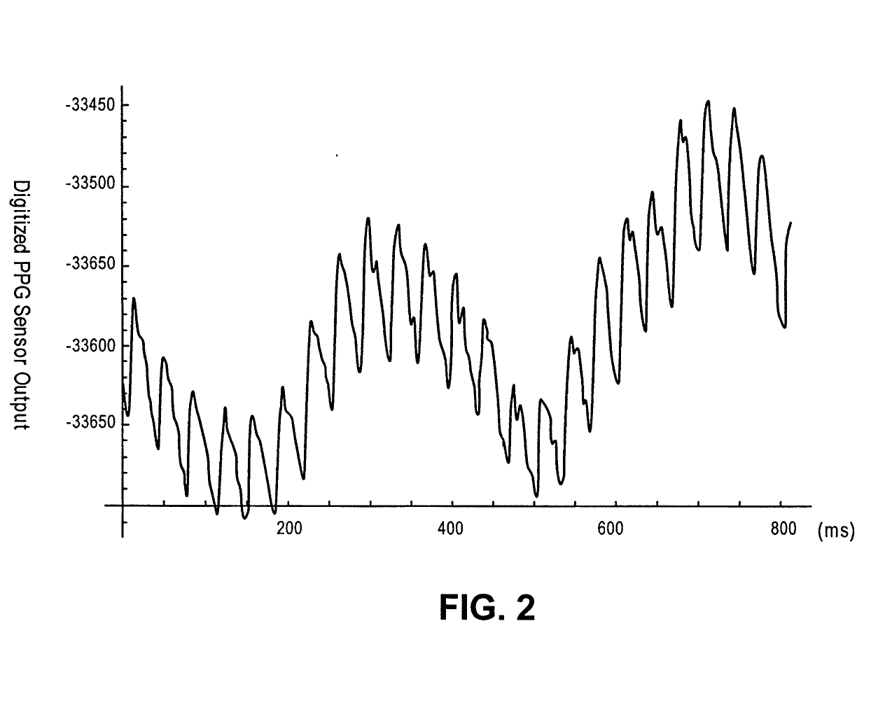

FIG. 2 demonstrates the typical multi-period PPG feature measured by our system. The presence of low frequency modulation and its expression is suggested in this invention as an indication of risk of cognitive distortions.

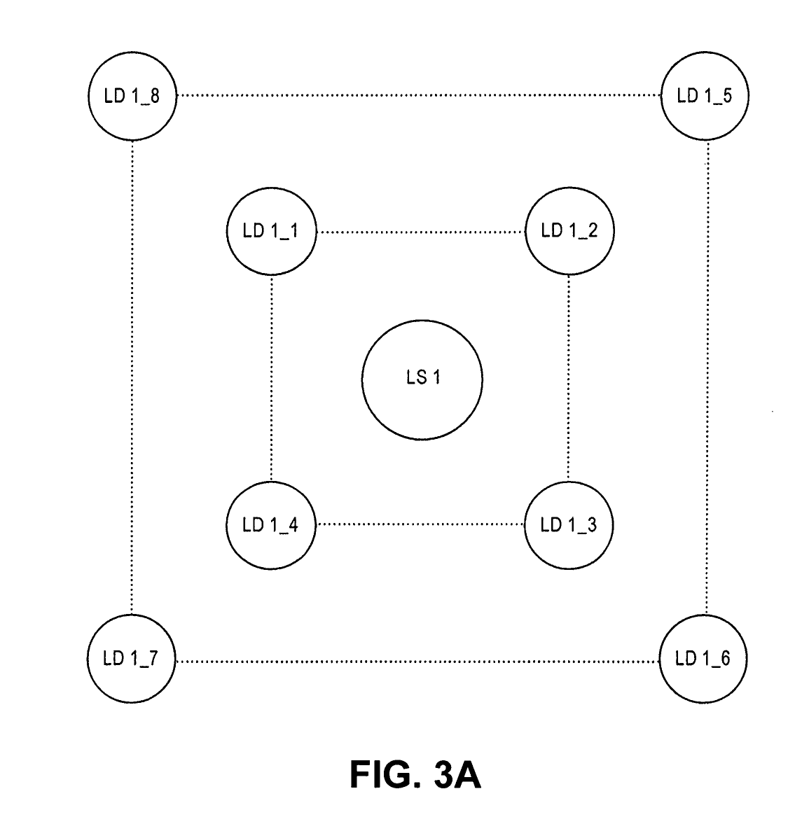

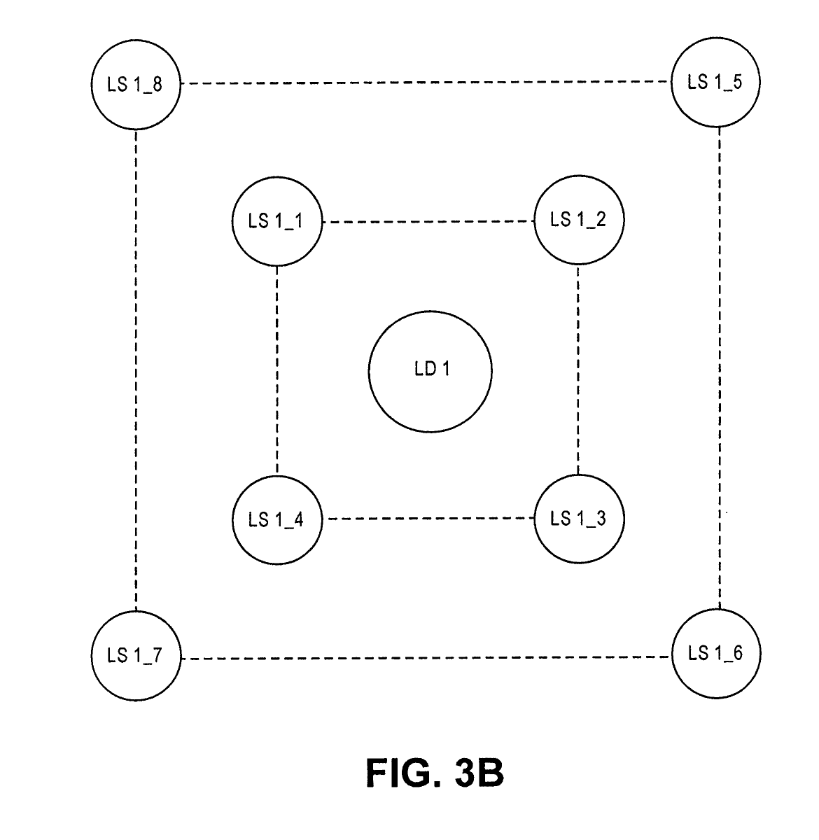

FIG. 3A demonstrates the layout of the one-wavelength part of our modified PPG unit. Such a layout provides in reflection the optical coupling between the light source and two sets of light detectors. All the detectors of each particular set, either the first set (detectors LD 1.1-LD1.4) or the second set (detectors LD1.5-LD1.8) have the same distance from the light source LS, while these distances for the first set and for the second set of detectors differ. The spacing between the light source and the various detectors may range from about 2 mm to about 20 mm. In one aspect, there is a spacing of about 3 mm for the first set in a spatial array and about 5 mm for the second set in a spatial array.

FIG. 3B demonstrates the layout of the one-wavelength part of a modified PPG unit. Such a layout provides in reflection the optical coupling between a single optical detector and two or more sets of optical light sources. All the light sources of each particular set, either the first set (sources LS 1_1-LS 1_4) or the second set (sources LS 1_5-LS 1_8) have the same distance from the light detector, while these distances for the first set and for the second set of sources differ. The spacing between the detector and the various emitters may range from about 2 mm to about 20 mm. In one aspect, there is a spacing of about 3 mm for the first set in a spatial array and about 5 mm for the second set in a spatial array.

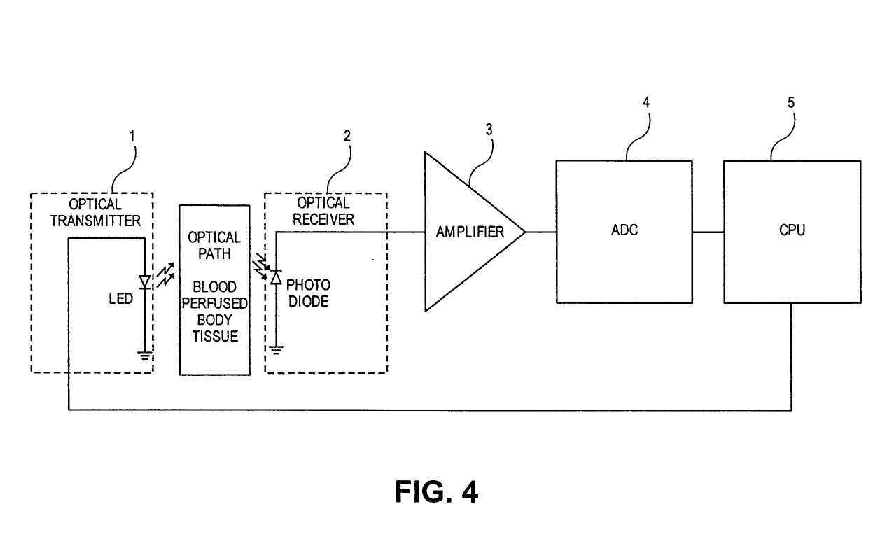

FIG. 4 is flow diagram showing the signal path from the optical transmitter to the CPU. It is to be appreciated that the proposed optical system is much smaller, energy efficient and may be placed on a finger, forehead, ear pinna, earlobe, cheek. In addition or optionally, the optical system may be inside the diver's mouth, in which case sensor may be mechanically combined with or integrated into a scuba mouthpiece. In addition or optionally, the optical system may be adapted and configured for placement along a portion of a diver's forehead in which case the sensor may be mechanically combined with or integrated into a dive mask. In other additional embodiments or configurations, there is provided a system that combines detection of decompression sickness with detection of such vital signs as heart rate, blood oxygen saturation (SpO2), respiration rate of a diver by analyzing photoplethysmogram (PPG) from a single miniature sensor and presents a significant improvement over existing ultrasound systems.

The optical transmitter 1 that includes an optical emitter comprises at least one light source configured for generating and applying an optical signal to a measurement location in a blood perfused body tissue. The optical receiver 2 includes an optical detector comprises at least one photodiode, configured for receiving light originated back from at least a portion of the measurement location and generating a photocurrent signal including response of the blood perfused body tissue to the optical signal. Signal amplifier 3 for amplification of the photocurrent signal generated by the optical detector 2. Analog to digital converter 4 to convert analog output of the signal amplifier 3 to digital domain for the further processing.

CPU 5 for processing the amplified and digitized photocurrent signal generated by the optical detector 2. The CPU 5 may include software, firmware or hardware for synchronized operation of one or more optical transmitters and/or received in accordance with the methods described herein.

The present invention can be attained by optical monitoring techniques that use light as an optical signal transmitted through a medium, such as a portion of a blood perfused body tissue for determining the vital signs and/or onset of or degree of decompression sickness. The optical detector converts the light (i.e., optical signal) into an analog electrical signal, which is subsequently amplified, digitized and provided to a CPU to retrieve information that was present in the optical signal. The information present in the optical signal may contain be both the information inserted by the transmitter as well as the information about the medium.

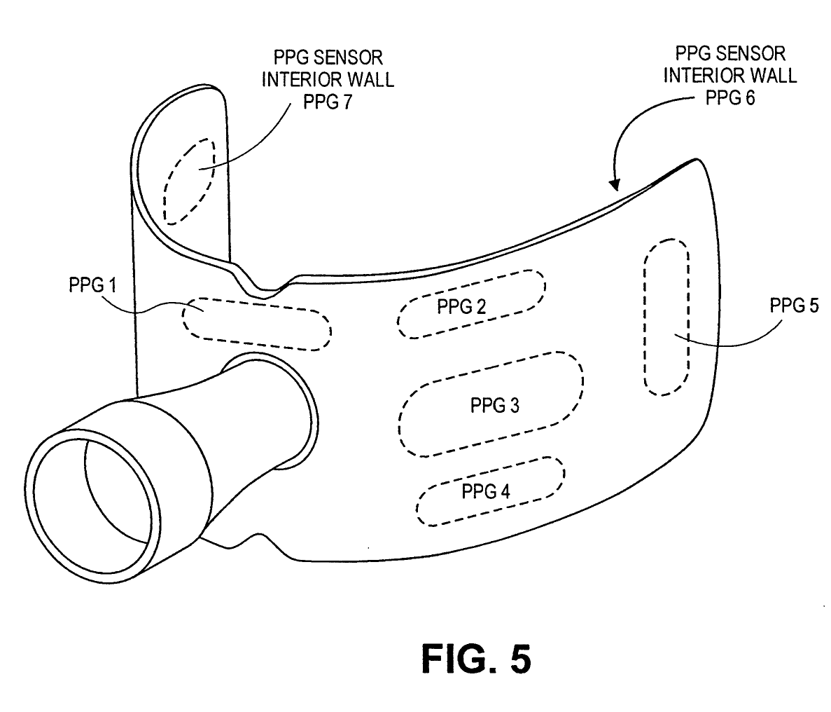

FIGS. 5-7 illustrate various locations where one or more PPG sensors may be mounted on or integrated to a portion of a mouth worn respirator. In one aspect, one or more PPG sensors are placed on, in or within a mouth worn respirator so that the PPG sensor may operate in transmission or reflectance mode with the vascular beds of the lips, gums, and/or cheeks in any combination.

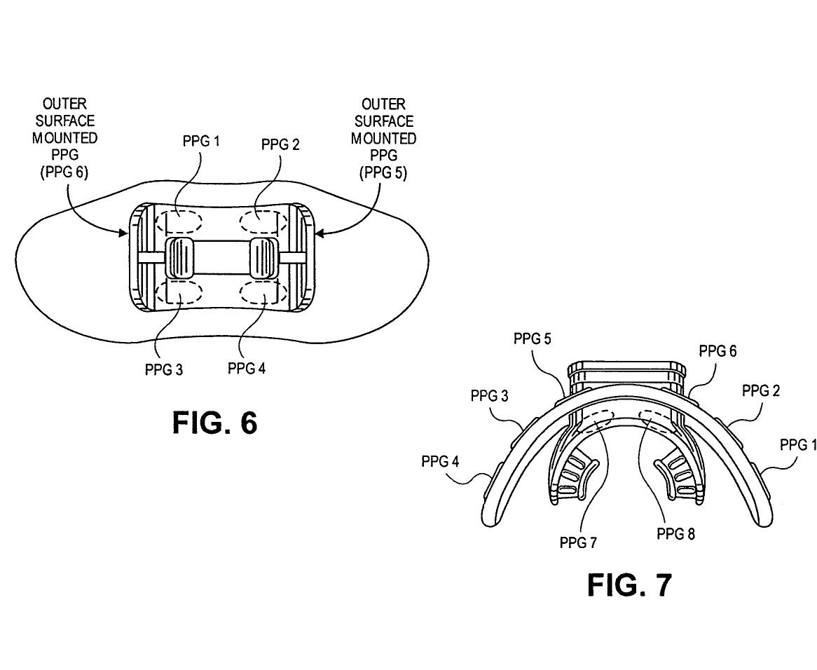

FIG. 5 is a perspective view of a respirator or SCUBA mouthpiece showing seven different exemplary PPG sensor mounting or integration locations. FIG. 6 is a perspective view of a respirator or SCUBA mouthpiece showing six different exemplary PPG sensor mounting or integration locations. FIG. 7 is a perspective view of a respirator or SCUBA mouthpiece showing eight different exemplary PPG sensor mounting or integration locations.

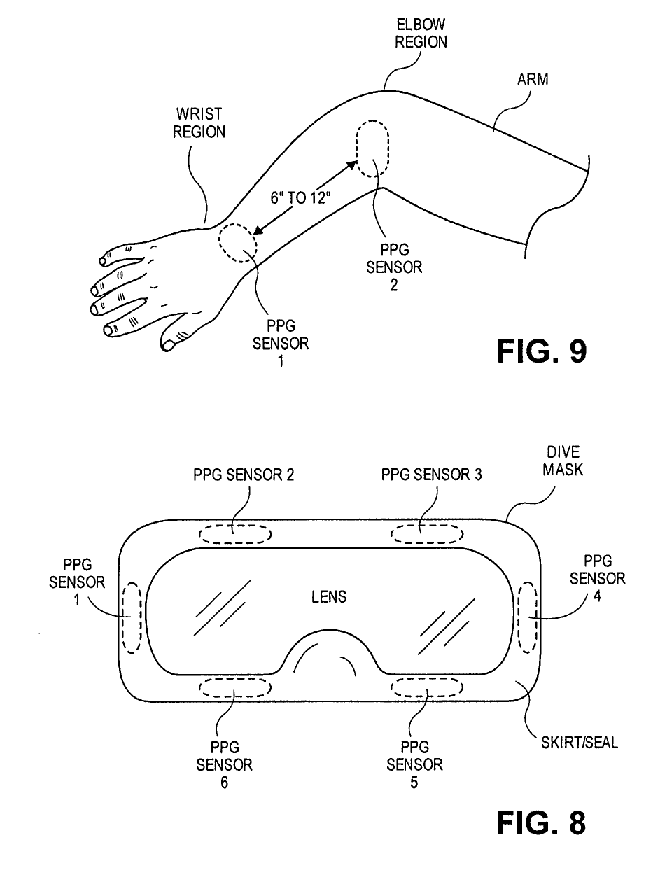

FIG. 8 is a rear view of the sealing rim of a dive mask showing six different exemplary PPG sensor mounting or integration locations. FIG. 8 illustrates various locations where one or more PPG sensors may be mounted on or integrated with a portion of a diving mask face piece or other sealing surface. In one aspect, one or more PPG sensors are placed on, in or within a sealing surface or skirt portion of a mask such that the PPG sensor is in contact with the skin of the diver as appropriate to a transmission or reflection PPG sensor configuration. It is to be appreciated that the mask born PPG sensor(s) may operate in transmission or reflectance mode with the vascular beds of the lips, forehead, temple or face, and/or cheeks in any combination.

FIG. 9 is a view of an arm of a diver show two spaced apart PPG sensor locations on the arm between about 6 inches to 12 inches apart. In the illustrated embodiment, there is one sensor near the wrist and another sensor near the elbow. The spacing of the sensors is particularly useful when analyzing PPG signal information for pulse wave comparisons as part of the mechanical indicia evaluation of detecting decompression sickness. While desiring not to be bound by theory, measuring blood speed at two different PPG sensors spaced apart permits comparison of differences in blood velocity from a reference blood speed of a user before a dive, during the descent stage of a dive, while at depth during a dive or during the ascent stage of a dive event. While desiring not to be bound by theory, if gas or bubbles are building up in the blood stream, then it is believed that the blood flow velocity will change. For example, the blood velocity information and comparison may reveal that the blood velocity is comparative slower than previously measured blood speed, then it may be an indication of the presence of bubbles or gas in the blood stream. In one aspect, comparisons of blood velocity from at least two PPG sensors on a user may be used to determine the presence or increasing presence or decreasing presence of gas or bubbles in the blood stream of the user.

One or more sensors with the same or different wavelength of the optical signal can be used in order to give a greater level of accuracy to the sensor readings. In one aspect, one or more PPG sensors may be operating in the green-yellow region (between 500-600 nm) while another may be operating in the red or infrared region. In another aspect, a suitable display for use in an underwater environment may be used which provides an output of PPG information related to one or more methods to the diver, or to another diver(s) or device which advises the diver, another diver or a safety monitor of the sensors analysis pertaining to the onset of or level of the caisson's disease and/or other vital signs. In another variation, if a radio is required then an appropriate radio technology might be integrated. The invention may be used for monitoring underwater diver's vital signs and stage of decompression sickness in real time for safety purposes.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements, these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A method of determining an initiation or progression of decompression sickness in an individual using an in-vivo measurement of time-dependence of optical density of at least one irradiated body part, the method comprising:
irradiating a portion of at least one body part with a photoplethysmography (PPG) sensor wherein the irradiating step is performed using at least one light source with the wavelength belonging to the red-near infrared (RNIR) spectral range or the yellow green spectral range;
collecting a time dependent optical signal from the sensor; and
analyzing the collected system to determine the initiation or progression of decompression sickness in the individual.

2. (canceled)

3. (canceled)

4. The method of claim 1 wherein the analyzing step is performed to determine an indicia of an optical scattering characteristic indicating the presence of a gas.

5. The method of claim 1 further comprising irradiating another portion of a body part spaced apart from the portion of at least one body part and wherein the analyzing step is performed by comparing a blood velocity measurement at the portion and the another portion to determine an indicia of the presence of a gas influencing blood velocity.

6. The method of claim 1 wherein the irradiated portions are spaced apart by a distance of 6 inches to 12 inches.

7. The method of claim 1 wherein the portion or the another portion of at least one body part is within the mouth, on a lip, on a cheek, on a forehead, on a face, on an ear, on a wrist, on an elbow, on an arm, on a leg, on a finger or on a hand.

8. The method of claim 1 wherein the irradiating step or the collecting step are performed using a PPG sensor wherein at least one element of an emitter or a detector is mounted on, in or within a respirator or SCUBA mouth piece or a diving mask.

9. The method of claim 1 wherein the irradiating step or the collecting step is performed using a PPG sensor configured as one emitter and multiple detectors.

10. The method of claim 1 wherein the irradiating step or the collecting step is performed using a PPG sensor configured as one detector and multiple emitters.

11. The method of claim 1 performed using at least one PPG sensor is configured for transmission mode operation.

12. The method of any of claim 1 performed using at least one PPG sensor is configured for reflectance mode operation.

13. A PPG sensor enabled diving mask, comprising:
a face piece having a lens and a seal or skirt; and
at least one PPG sensor on, in or within a portion of the seal or the skirt such that in use on a diver the at least on PPG sensor is positioned to irradiate a desired portion of a region of blood perfusion on the diver;

14. The diving mask of claim 11 wherein the at least one PPG sensor is adapted and configured to irradiate a desired portion using a light in the yellow-green region.

15. The diving mask of claim 11 wherein the at least one PPG sensor is adapted and configured to irradiate a desired portion using a light in the near infrared region.

16. The diving mask of claim 11 wherein the at least one PPG sensor is configured as one emitter and multiple detectors.

17. The diving mask of claim 11 wherein the at least one PPG sensor is configured as one detector and multiple emitters.

18. The diving mask of claim 11 wherein the at least one PPG sensor is configured for transmission mode operation.

19. The diving mask of claim 11 wherein the at least one PPG sensor is configured for reflectance mode operation.

20. A PPG sensor enabled respirator mouthpiece, comprising:
a respirator mouthpiece having a connector to attachment to a respirator supply and a portion for placement within the mouth of a user having an inner surface and an outer surface; and
at least one PPG sensor on, in or within a portion of the respirator mouthpiece positioned to irradiate a desired portion of a region of blood perfusion on the user having the mouthpiece in his mouth.
